(19) United States
(12) Patent Application Publication
(10) Pub. No.: US 2008/0020375 A1
(43) Pub. Date: Jan. 24, 2008

(54) Method Of Adjudicating On Prostate Cancer

(76) Inventors: Hiroshi Yamamoto, Suita-shi (JP); Noboru Konishi, Kashihara-shi (JP); Kazutake Tsujikawa, Suita-shi (JP)

Correspondence Address: WENDEROTH, LIND & PONACK, L.L.P., 2033 K STREET N. W., SUITE 800, WASHINGTON, DC, 20006-1021, US

(21) Appl. No.: 10/590,146

(22) PCT Filed: Aug. 24, 2004
(86) PCT No.: PCT/JP04/12446

§ 371 Date: Jun. 8, 2007

(51) Int. Cl. C12Q 1/68; C07H 21/04; C07K 1/00; C07K 16/18

(52) U.S. Cl. 435/6; 530/350; 530/389.1; 536/23.5

(57) ABSTRACT

The present invention relates to a method for determining a subject having a risk of having developed or developing prostate cancer, which comprises the following steps: a) a step of analyzing the presence/absence or level of mutation in the PCA-1 gene derived from the subject; and b) a step of evaluating the presence/absence or level of the subject's risk of having developed or developing prostate cancer, based on the presence/absence or level of mutation in the PCA-1 gene. According to the determination method of prostate cancer of the present invention, a subject having a risk of having developed or developing prostate cancer can be determined conveniently and highly sensitively. Therefore, the method is effective for the diagnosis of prostate cancer, progress monitoring, prognostic prediction, diagnosis before the onset, carrier diagnosis and the like.

### TECHNICAL FIELD

The present invention relates to a method of determining prostate cancer. More particularly, the present invention relates to a method of determining a subject having a risk of having developed or developing prostate cancer, which comprises the following steps:
a) a step of analyzing the presence/absence or level of mutation in the PCA-1 gene derived from the subject; and
b) a step of evaluating the presence/absence or level of the subject's risk of having developed or developing prostate cancer based on the presence/absence or level of mutation in PCA-1 gene.

### BACKGROUND ART

Prostate cancer is a malignant tumor that tops the list of male cancer morbidity rates and mortality rates in the US and Europe. In Japan, too, the morbidity rate and mortality rate of prostate cancer have been rapidly increasing in recent years with the westernization of the dietary habits and aging.

At present, the diagnosis of prostate cancer depends on histopathological evaluations such as tumor grade, stage and the like and biochemical parameters such as prostate specific antigen (PSA) value and the like. Particularly, measurement of a PSA concentration in blood has rapidly become pervasive in mass health screening and complete medical check, which has enabled discovery of prostate cancer in comparatively early stages. However, the PSA concentration in blood increases only after the onset of prostate cancer, and it is sometimes difficult to distinguish prostate cancer from prostatomegaly (benign prostatic hyperplasia) or prostatitis by the diagnosis based on the PSA concentration in blood. Therefore, the development of a more specific and highly sensitive new marker for the detection, diagnosis or progress monitoring of prostate cancer has been desired.

The present inventors histopathologically separated a prostate tissue removed from a prostate cancer patient into a tumor site and a non-tumor site, and analyzed a gene showing different expression amounts between a non-tumor site and a tumor site, for the purpose of seeking a treatment target molecule of prostate cancer. As a result, they have succeeded in cloning a gene called PCA-1 (also called human AlkB homolog 3: hABH3) (the 123rd Annual Meeting of The Pharmaceutical Society of Japan, Abstract 4, p. 15 (2003)) as a gene specifically and highly expresses in a tumor site as compared to a non-tumor site. In recent years, PCA-1 has been confirmed to be a DNA, RNA alkylation damage repair enzyme, and its function has been drawing attention (Nature, vol. 421, pp. 859-863 (2003)).

In view of the above-mentioned situation, the present invention aims at provision of a new method of determining a subject having a risk of having developed or developing prostate cancer.

### DISCLOSURE OF THE INVENTION

The present inventors have conducted intensive studies in an attempt to achieve the above-mentioned object and found that, in contrast to the assumed presence of some causal association between promotion of PCA-1 function and the onset or progress of prostate cancer, since expression of PCA-1 gene increases in a prostate cancer tissue, mutation in PCA-1 gene that could cause dysfunction of PCA-1 is observed at high frequency in prostate cancer patients, which resulted in the completion of the present invention.

Accordingly, the present invention relates to the following (1)-(19).
(1) A method for determining a subject having a risk of having developed or developing prostate cancer, which comprises the following steps:
a) a step of analyzing the presence/absence or level of mutation in the PCA-1 gene derived from the subject; and
b) a step of evaluating the presence/absence or level of the subject's risk of having developed or developing prostate cancer, based on the presence/absence or level of mutation in the PCA-1 gene.
(2) The method of the above-mentioned (1), wherein the mutation is selected from the group consisting of a missense mutation, a nonsense mutation, a silent mutation and a frame shift mutation due to deletion.
(3) The method of the above-mentioned (2), wherein the missense mutation causes an amino acid mutation selected from the group consisting of mutation of the 7th arginine to leucine, mutation of the 8th alanine to valine, mutation of the 30th alanine to threonine, mutation of the 41st threonine to isoleucine, mutation of the 73rd aspartic acid to asparagine, mutation of the 137th glycine to arginine, mutation of the 144th serine to proline, mutation of the 228th aspartic acid to glutamic acid, mutation of the 233rd glutamic acid to aspartic acid, and mutation of the 261st lysine to asparagines, in PCA-1 polypeptide.
(4) The method of the above-mentioned (3), wherein the missense mutation causes an amino acid mutation selected from the group consisting of mutation of the 228th aspartic acid to glutamic acid, mutation of the 233rd glutamic acid to aspartic acid, and mutation of the 261st lysine to asparagines, in PCA-1 polypeptide.
(5) The method of the above-mentioned (4), wherein the missense mutation causes a mutation of the 228th aspartic acid to glutamic acid in PCA-1 polypeptide.
(6) The method of the above-mentioned (3), wherein the missense mutation is selected from the group consisting of mutation of the 426th guanine to thymine, mutation of the 29th cytosine to thymine, mutation of the 494th guanine to adenine, mutation of the 528th cytosine to thymine, mutation of the 623rd guanine to adenine, mutation of the 815th guanine to adenine, mutation of the 836th thymine to cytosine, mutation of the 1090th cytosine to guanine, mutation of the 1105th adenine to thymine, and mutation of the 1189th adenine to thymine, in PCA-1 gene.
(7) The method of the above-mentioned (6), wherein the missense mutation is selected from the group consisting of mutation of the 1090th cytosine to guanine, mutation of the 1105th adenine to thymine, and mutation of the 1189th adenine to thymine, in PCA-1 gene.
(8) The method of the above-mentioned (7), wherein the missense mutation is mutation of the 1090th cytosine to guanine in PCA-1 gene.
(9) The method of the above-mentioned (2), wherein the nonsense mutation causes mutation of the 261st lysine to a stop codon in PCA-1 polypeptide.
(10) The method of the above-mentioned (9), wherein the nonsense mutation is mutation of the 1187th adenine to thymine in PCA-1 gene.
(11) The method of the above-mentioned (2), wherein the silent mutation is selected from the group consisting of mutation of the 568th thymine to cytosine and mutation of the 1132nd guanine to thymine, in PCA-1 gene.
(12) The method of the above-mentioned (11), wherein the silent mutation is mutation of the 1132nd guanine to thymine in PCA-1 gene.
(13) The method of the above-mentioned (2), wherein the deletion is a deletion of the residue from the 777th adenine to the 865th cytosine in PCA-1 gene.
(14) The method of the above-mentioned (1), which comprises the following steps:
a) a step of isolating or purifying a polynucleotide from the subject;
b) a step of amplifying a PCA-1 gene from the polynucleotide;
c) a step of determining the base sequence of the amplified PCA-1 gene;
d) a step of analyzing the presence/absence or level of mutation in the PCA-1 gene derived from the subject; e) a step of evaluating the presence/absence or level of the subject's risk of having developed or developing prostate cancer, based on the presence/absence or level of mutation in the PCA-1 gene.
(15) A polynucleotide comprising a base sequence having not less than one substitution or deletion selected from the group consisting of substitution to the 426th guanine to thymine, substitution of the 429th cytosine by thymine, substitution of the 494th guanine by adenine, substitution of the 528th cytosine by thymine, substitution of the 623rd-guanine by adenine, substitution of the 815th guanine by adenine, substitution of the 836th thymine by cytosine, substitution of the 1090th cytosine by guanine, substitution of the 1105th adenine by thymine, substitution of the 1189th adenine by thymine, substitution of the 1187th adenine by thymine, substitution of the 568th thymine by cytosine, substitution of the 1132nd guanine by thymine, and deletion of a sequence from the 777th adenine to the 865th cytosine, as compared to SEQ ID; No 1.
(16) A polypeptide comprising an amino acid sequence encoded by a base sequence having not less than one substitution or deletion selected from the group consisting of substitution to the 426th guanine to thymine, substitution of the 429th cytosine by thymine, substitution of the 494th guanine by adenine, substitution of the 528th cytosine by thymine, substitution of the 623rd guanine by adenine, substitution of the 815th guanine by adenine, substitution of the 836th thymine by cytosine, substitution of the 1090th cytosine by guanine, substitution of the 1105th adenine by thymine, substitution of the 1189th adenine by thymine, substitution of the 1187th adenine to thymine, and deletion of a sequence from the 777th adenine to the 865th cytosine, as compared to SEQ ID; No 1.
(17) An antibody immunospecifically recognizing the polypeptide of the above-mentioned (16).
(18) A kit for determining a subject having a risk of having developed or developing prostate cancer, which comprises a reagent for PCA-1 gene mutation analysis.
(19) The kit of the above-mentioned (17), further comprising a written matter associated therewith, the written matter stating that the reagent for PCA-1 gene mutation analysis can or should be used for determining a subject having a risk of having developed or developing prostate cancer.

According to the determination method of prostate cancer of the present invention, since a subject having a risk of having developed or developing prostate cancer can be determined conveniently and highly sensitively, it is effective for the diagnosis, progress monitoring, prognostic prediction, diagnosis before the onset, carrier diagnosis and the like of prostate cancer.

### BRIEF DESCRIPTION OF THE DRAWINGS

FIG. 1 is a graph showing the relationship between the MMS concentration and the number of adhered cells, wherein the vertical axis shows a relative value of the number of adhered cells based on the control group as 100%, the axis of abscissas shows-MMS concentration (mM), the black column shows the control group, the hatched column shows a PCA-1 WT transfection group and the white column shows a PCA-1A777-865 transfection group.

### DETAILED DESCRIPTION OF THE INVENTION

In the present invention, the “prostate cancers is a conception widely encompassing cancer developed in the prostate and encompasses not only adenocarcinoma developed in the prostate but also squamous cell carcinoma, transitional cell carcinoma, neuroendocrine carcinoma, undifferentiated cancer and the like. The prostate cancer is preferably an adenocarcinoma developed in the prostate.

In humans, PCA-1 is a molecule called human AlkB homolog 3 (hABH3) or DEPC-1.

As a base sequence (cDNA base sequence) of normal human PCA-1 gene, for example, SEQ ID; No 1 and the like can be mentioned, wherein the 407th -1267th residue (SEQ ID; No 3) corresponds to a protein coding region that encodes PCA-1 polypeptide having the amino acid sequence shown in SEQ ID; No 2. In the present invention, the position of the residue in the base sequence and amino acid sequence in PCA-1 gene is based on the sequences shown in the above-mentioned SEQ ID; Nos 1 and 2, respectively. Here, the 923rd-1243rd residues of SEQ ID; No 1 and the 172nd-279th amino acid residues of SEQ ID; No 2 correspond to the dealkylation enzyme active site of PCA-1.

Human PCA-1 gene is coded on p11 locus of the 11th chromosome, and PCA-1 gene and chromosomal DNA base sequence around the gene are disclosed, for example, under GenBank Accession No. NT_{—}009237 (NCBI homepage) and the like.

Human PCA-1 gene contains 10 exons, and in SEQ ID; No 1, exon 1 corresponds to the 1st-334th residues, exon 2 corresponds to the 335th-485th residues, exon 3 corresponds to the 486th-592nd residues, exon 4 corresponds to the 593rd-624th residues, exon 5 corresponds to the 625th-670th residues, exon 6 corresponds to the 671st-776th residues, exon 7 corresponds to the 777th-865th residues, exon 8 corresponds to the 866th-1075th residues, exon 9 corresponds to the 1076th-1172nd residues, and exon 10 corresponds to the 1173rd-1520th residues.

The determination method of prostate cancer of the present invention comprises at least the following steps:
a) a step of analyzing the presence/absence or level of mutation in the PCA-1 gene derived from the subject; and
b) a step of evaluating the presence/absence or level of the subject's risk of having developed or developing prostate cancer, based on the presence/absence or level of mutation in the PCA-1 gene.

In the step of analyzing the presence/absence or level of mutation in the PCA-1 gene derived from the subject, the PCA-1 gene derived from the subject is compared with the normal PCA-1 gene.

The comparison between a PCA-1 gene derived from a subject and a normal PCA-1 gene is not particularly limited and, for example, is a comparison with a base sequence of mRNA, cDNA, chromosome DNA and the like of a normal PCA-1 gene.

Here, the “comparison with a base sequence of a normal PCA-1 gene” may be, besides a comparison with the full-length base sequence of a normal PCA-1 gene, a comparison with a complementary sequence, a partial sequence, a complementary sequence of a partial sequence, a sequence of particular residues and the like of the sequence.

Here, the length of the above-mentioned partial sequence is not less than 10 bases, preferably not less than 30 bases. Preferable embodiments of the partial sequence include a base sequence of a protein coding region of PCA-1, and a base sequence of the exon region in PCA-1 gene can be mentioned.

For the analysis of the presence/absence or level of mutation in PCA-1 gene derived from the subject, any method known per se can be used.

For example, the base sequence of PCA-1 gene in the polynucleotide derived from the subject may be determined. In addition, any methods currently used in the pertinent field for detecting changes in the base, such as RFLP method, TaqMan test, oligonucleotideligase method, test methods based on single chain conformation polymorphism or hybridization of template nucleic acid to oligonucleotide array, denatured gradient gel electrophoresis, PCR-SSPC method, Southern blotting, Northern blotting, ASO method, ARMS method using a mismatch primer and the like, can also be used. Furthermore, methods to be developed in the future can also be used as methods for analyzing the presence or absence of mutation based on the above-mentioned comparison. Preferably, the base sequence of PCA-1 gene in the polynucleotide derived from the subject is determined.

For determination of the base sequence in PCA-1 gene in the polynucleotide derived from the subject, polynucleotide is first isolated and purified from a subject. The polynucleotide preferably contains a PCA-1 gene derived from the subject.

The polynucleotide is isolated and purified, for example, from a sample derived from the subject, such as a tissue, a cell, a liquid component and the like.

While the tissue is not particularly limited, it is, for example, prostate, hair, skin and the like, with preference given to prostate.

The cell includes both a cell separated from a subject, which has not been cultured and a cell separated from a living organism, which has been cultured. For example, a blood cell, a prostate-derived cell and the like can be mentioned, with preference given to a prostate-derived cell.

As the liquid component, for example, blood, sperm, saliva, urine, sweat and the like can be mentioned.

As the polynucleotide, for example, DNA (chromosome DNA, cDNA etc.) and RNA (total RNA, mRNA, cRNA etc.) can be mentioned, with preference given to chromosome DNA, total RNA or mRNA.

Polynucleotide can be isolated or purified by a method known per se.

For isolation or purification of chromosome DNA, for example, proteinase K/phenol extraction method, proteinase K/phenol/chloroform extraction method, alkali dissolution method, boiling method and the like can be used.

For isolation or purification of total RNA, for example, guanidine-cesium chloride ultracentrifugation method, AGPC method (Acid Guanidinium-Phenol-Chloroform method) and the like can be used. Alternatively, commercially available kits such as TRIzol (manufactured by Life Technologies, Inc.), Isogen (manufactured by Nippon Gene) and the like can also be used. mRNA can be isolated or purified by applying purified total RNA to oligo dT column and the like.

When polynucleotide is RNA, particularly total RNA or mRNA, it is preferable to synthesize cDNA by a method known per se utilizing reverse transcriptase and the like.

For determination of the base sequence of PCA-1 gene in the polynucleotide, the PCA-1 gene in the polynucleotide is preferably amplified using the polynucleotide as a template. While the polynucleotide to be used as a template is not particularly limited, it is preferably chromosome DNA or cDNA.

While the method of gene amplification is not particularly limited, polymerase chain reaction (PCR), LAMP (Loop-mediated isothermal amplification) (e.g., see WO 00/28082), ICAN (Isothermal and Chimeric primer-initiated Amplification of Nucleic acids) (e.g., see WO 00/56877), self-sustained sequence replication (Guatelli et al., Proc. Natl. Acad. Sci. USA, 87:1874-1878 (1990)), Transcription-based amplification system (Kwoh et al., Proc. Natl. Acad. Sci. USA, 86:1173-1177 (1989)), Q-β replicase (Lizardi et al., Bio/Technology, 6:1197 (1988)) and the like can be mentioned. While any of them can be used, PCR is convenient and preferable. Reference literatures for general use of PCR technique include Mullis et al., Cold Spring Harbor Symp. Quant. Biol., 51:263 (1987), Ehrlich(ed), PCR technology, Stockton Press, NY, 1989, Ehrlich et al., Science, 252: 1643-1650 (1991), PCR protocols; A Guide to Methods and Applications, Eds. Innis et al., Academic Press, New York (1990) and the like.

As polymerase to be used for amplification of PCA-1 gene by PCR, any of Taq polymerases and the like generally used by those of ordinary skill in the art can be used. Preferably, a polymerase showing comparatively high fidelity, such as KOD Plus (manufactured by TOYOBO) and the like are used.

For amplification of PCA-1 gene by PCR, an oligonucleotide primer set that enables amplification of the gene is used. The primer set can be appropriately designed by those of ordinary skill in the art based on, for example, the information in the present DESCRIPTION such as the base sequence shown in SEQ ID; No 1 and the like, the base sequence of p11 region of human 11th chromosome and the like disclosed in GenBank Accession No. NT_{—}009237 (NCBI homepage) and the like, and using an appropriate program such as Oligo 4.0.6 (manufactured by National Bioscience, Plymouth Minn.) and the like, and the like.

When the template for PCR is cDNA, while the region where the primer set is designed is not particularly limited, the primer set is preferably designed such that it can amplify at least one, more preferably two or more, most preferably all of the residues selected from the group consisting of the residues of the 426th guanine, the 429th cytosine, the 494th guanine, the 528th cytosine, the 568th thymine, the 623rd guanine, the 815th guanine, the 836th thymine, the 1090th cytosine, the 1105th adenine, the 1132nd guanine, the 1187th adenine, the 1189th adenine, and the 777th adenine to the 865th cytosine of SEQ ID; No 1.

When the template for PCR is chromosome DNA, moreover, while the region where the primer set is designed is not particularly limited, the primer set is preferably designed such that it can amplify exon region, more preferably designed such that it can amplify at least one, more preferably two or more, most preferably all of the residues on chromosome DNA corresponding to the aforementioned residues. Plural primer sets may be set.

While the size of the oligonucleotide to be used as a primer can be appropriately determined, it is generally 10-100 bp, preferably 12-50 bp, more preferably 15-30 bp.

While the GC content of the oligonucleotide to be used as a primer can be appropriately determined, it is generally within the range of 20-80%, preferably 30-70%, more preferably 40-60%.

The Tm value of the oligonucleotide to be used as a primer is generally set to fall within the range of 60±20° C. While the Tm value varies depending on the salt concentration and the like in the buffer, it can be calculated according to a method known per se, such as a nearest neighbor method (e.g., see “Biochemistry, 35, 3555-3562, 1996” and the like) and the like.

Oligonucleotide may be naturally occurring or synthesized, and can be synthesized by phosphotriethyl method, phosphodiester method and the like, using a conventional nucleic acid autosynthesizer.

The primer is designed to produce an amplification product having a size of generally about 50-30000 bp. In consideration of the amplification efficiency and the like, the size of the amplification product is preferably set to about 50-3000 bp, more preferably about 100-2000 bp.

As a preferable primer set, for example, a primer set of

| | | |
|---|---|---|
| primer 1: | CTGAAAGCTCGGAGCAGAAGC | (SEQ ID; No 4) |
| primer 2: | GGTCTACTGTGGGAACAG | (SEQ ID; No 5) |

and the like can be mentioned.

The base sequence of the amplified PCA-1 gene can be determined by a well-known method. As a preferable method, methods based on incorporation of terminal nucleotide into a template polymerase synthesized copy by the method of Sanger and the like can be mentioned. As an alternative method, for example, adapter sequence determination (PCT/US95/12678), sequence determination based on ligation (PCT/US96/05245), sequence determination by hybridization (A. D. Mirzabekov, TIBTech 12:27-32, 1994) and the like can be mentioned.

Based on the determined base sequence, the presence/absence or level of mutation of a PCA-1 gene derived from a subject can be analyzed.

When the template used in the above-mentioned amplification is a chromosome DNA, a base sequence contained in the exon region of the PCA-1 gene in the determined base sequence is preferably compared with the corresponding base sequence in the cDNA base sequence of a normal PCA-1 gene. In addition, when the template used in the above-mentioned amplification is a cDNA, the determined base sequence is compared with the corresponding base sequence in the cDNA base sequence of a normal PCA-1 gene.

When a mutation is detected in a PCA-1 gene derived from a subject, i.e., when one or more residual bases different from a normal PCA-1 gene are detected in the analyzed PCA-1 gene derived from a subject, the subject is determined to have a risk (or a high risk) of having developed or developing prostate cancer. Conversely, when a mutation is not detected in a PCA-1 gene derived from a subject, the subject is determined to have no or a low risk of having developed or developing prostate cancer.

According to the method of the present invention, a higher level of mutation can be considered to show a subject's higher risk of having developed or developing prostate cancer. By the “level of mutation” is meant, for example, the number of mutated sites, the length of the base involved in the mutation and the like.

When a mutation of PCA-1 gene is present in one of the alleles of the PCA-1 gene derived from a subject, the subject is considered to have a risk of having developed or developing prostate cancer. When a mutation is found in both alleles, a higher risk can be considered as compared to the presence of a mutation in only one of the alleles.

When superiority or inferiority is found in alleles, a mutation in dominant allele can-be considered to show a higher risk as compared to the presence of a mutation in recessive allele.

When the method of the present invention is used in combination with other diagnosis method of prostate cancer, the precision of the determination can be enhanced. As other diagnosis method of prostate cancer, palpation test, methods using prostate specific antigen (PSA) concentration in serum and the like, pathology tests using prostate needle biopsy samples, ultrasonography, MRI, CT, bone scintigraphy and the like can be mentioned. In addition, the method of the present invention can also be used in combination with test methods using, as an index, an increased expression of PCA-1 gene in a prostate cancer tissue (see non-patent reference 1).

The mutation site of the above-mentioned PCA-1 gene is not particularly limited.

In chromosome DNA, the mutation site may be in any of the regions encoding the PCA-1 gene (e.g., enhancer region, promoter region, exon region, intron region etc.). Preferred is an exon region or an intron region, and more preferred is an exon region.

In cDNA or mRNA, the mutation site of the PCA-1 gene is preferably in a protein coding region.

The kind of mutation of the above-mentioned PCA-1 gene is not particularly limited and, for example, a mutation due to substitution such as a missense mutation, a nonsense mutation, a silent mutation and the like, a frame shift mutation due to deletion, insertion and the like, and the like can be mentioned. Preferred mutation is selected from the group consisting of a missense mutation, a nonsense mutation, a silent mutation and a frame shift due to deletion.

An embodiment of amino acid mutation in the missense mutation in PCA-1 gene is not particularly limited. Preferably, the missense mutation is a mutation causing a mutation of an amino acid residue selected from the group consisting of the 7th arginine, the 8th alanine, the 30th alanine, the 41st threonine, the 73rd aspartic acid, the 137th glycine, the 144th serine, the 228th aspartic acid, the 233rd glutamic acid, and the 261st lysine in PCA-1 polypeptide.

In this case, the kind of the amino acid after the substitution is not particularly limited. Preferably, the missense mutation is a mutation causing amino acid mutation selected from the group consisting of mutation of the 7th arginine to leucine, mutation of the 8th alanine to valine, mutation of the 30th alanine to threonine, mutation of the 41st threonine to isoleucine, mutation of the 73rd aspartic acid to asparagine, mutation of the 137th glycine to arginine, mutation of the 144th serine to proline, mutation of the 228th aspartic acid to glutamic acid, mutation of the 233rd glutamic acid to aspartic acid, and mutation of the 261st lysine to asparagine in PCA-1 polypeptide.

More preferably, the missense mutation is a mutation causing amino acid mutation in dealkylation enzyme active site in PCA-1 polypeptide (corresponding to the 172-279th amino acid residues of PCA-1 polypeptide), i.e., a mutation causing amino acid mutation selected from the group consisting of mutation of the 228th aspartic acid to glutamic acid, mutation of the 233rd glutamic acid to aspartic acid, and mutation of the 261st lysine to asparagine.

Further preferably, the missense mutation is a mutation causing mutation of the 228th aspartic acid to glutamic acid in PCA-1 polypeptide.

In the above-mentioned missense mutation, a base sequence encoding the amino acid after mutation is not particularly limited. Preferably, the missense mutation is a mutation selected from the group consisting of mutation of the 426th guanine to thymine, mutation of the 429th cytosine to thymine, mutation of the 494th guanine to adenine, mutation of the 528th cytosine to thymine, mutation of the 623rd guanine to adenine, mutation of the 815th guanine to adenine, mutation of the 836th thymine to cytosine, mutation of-the 1090th cytosine to guanine, mutation of the 1105th adenine to thymine, and mutation of the 1189th adenine to thymine in PCA-1 gene.

While the embodiment of the amino acid mutation in the nonsense mutation in PCA-1 gene is not particularly limited, the nonsense mutation is preferably a mutation causing mutation of the 261st lysine to a stop codon in PCA-1 polypeptide.

In the nonsense mutation, the base sequence encoding the stop codon after mutation is not particularly limited. Preferably, the nonsense mutation is mutation of the 1187th adenine to thymine in the PCA-1 gene base sequence.

While the mutated site in the silent mutation in PCA-1 gene is not particularly limited, the silent mutation is preferably a mutation of a residue selected from the group consisting of the 568th thymine and the 1132nd guanine in PCA-1 gene, and more preferably, a mutation of the 1132nd guanine.

In the silent mutation, the base after the mutation is not particularly limited. Preferably, the silent mutation is a mutation selected from the group consisting of mutation of the 568th thymine to cytosine and mutation of the 1132nd guanine to thymine, in PCA-1 gene.

In the deletion of PCA-1 gene, the number, position etc. of the base to be deleted is not particularly limited. Preferably, the deletion is deletion of one or multiple exon regions, more preferably, deletion of one exon region.

While the exon region to be deleted is not particularly limited, in the case of a human PCA-1 gene, it is preferably exon 7 (the 777th-865th residues in SEQ ID; No 1).

Since the deletion causes substitution of amino acids beginning from the 124th amino acids in PCA-1 polypeptide, as well as substitution of the 136th amino acid by a stop codon, the protein translation ends at the 135th amino acid.

Moreover, the present invention relates to a polynucleotide comprising a base sequence having not less than one substitution or deletion selected from the group consisting of substitution of the 426th guanine by thymine, substitution of the 429th cytosine by thymine, substitution of the 494th guanine by adenine, substitution of the 528th cytosine by thymine, substitution of the 623rd guanine by adenine, substitution of the 815th guanine by adenine, substitution of the 836th thymine by cytosine, substitution of the 1090th cytosine by guanine, substitution of the 1105th adenine by thymine, substitution of the 1189th adenine by thymine, substitution of the 1187th adenine by thymine, substitution of the 568th thymine by cytosine, substitution of the 1132nd guanine by thymine, and deletion of a sequence from the 777th adenine to the 865th cytosine, as compared to SEQ ID; No 1. Preferably,-the polynucleotide-is a polynucleotide having a base sequence containing one or more substitutions selected from the above-mentioned group, as compared to SEQ ID; No 1.

In addition, the present invention encompasses a polynucleotide which is a part of the above-mentioned polynucleotide and which has not less than one substitution or deletion selected from the above-mentioned group, as compared to SEQ ID; No 1. The length of the part of polynucleotide is not less than 10 bp, preferably not less than 30 bp.

The above-mentioned polynucleotide of the present invention is useful in the analysis of mutation in human PCA-1 gene, for example, as a reagent such as a mismatch primer, a mismatch probe and the like, or a polynucleotide control already clarified to contain a PCA-1 gene having a mutation.

Moreover, by introducing a suitable expression vector functionally inserted with the polynucleotide of the present invention into a host cell such as prostate cancer cell and the like, allowing expression of the mutated PCA-1 polypeptide, analyzing the host cell, and the like, the relationship between the mutation in PCA-1 gene and prostate cancer can be studied. Accordingly, the polynucleotide of the present invention is useful as a reagent for the research of prostate cancer.

The polynucleotide of the present invention is preferably isolated or purified.

The present invention relates to a polypeptide containing an amino acid sequence encoded by the base sequence having not less than one substitution or deletion selected from the group consisting of substitution of the 426th guanine by thymine, substitution of the 429th cytosine by thymine, substitution of the 494th guanine by adenine, substitution of the 528th cytosine by thymine, substitution of the 623rd guanine by adenine, substitution of the 815th guanine by adenine, substitution of the 836th thymine by cytosine, substitution of the 1090th cytosine by guanine, substitution of the 1105th adenine by thymine, substitution of the 1189th adenine by thymine, substitution of the 1187th adenine by thymine, and deletion of a sequence from the 777th adenine to the 865th cytosine, as compared to SEQ ID; No 1. Preferably, the polypeptide is a polypeptide having an amino acid sequence encoded by a base sequence having not less than one substitution or deletion selected from the above-mentioned group, as compared to SEQ ID; No 1.

In addition, the present invention encompasses a polypeptide which is a part of the above-mentioned polypeptide, which contains at least one amino acid substituted by the above-mentioned group of substitution or deletion. The length of the partial polypeptide is not less than 6 amino acids, preferably not less than 8 amino acids.

The polypeptide of the present invention is preferably isolated or purified.

The polypeptide of the present invention is useful as an immunogen for the production of an antibody that immunospecifically recognizes a polypeptide of human PCA-1 having the above-mentioned mutation. The present invention also relates to an antibody.

By the “immunospecifically” is meant that an antibody shows a substantially higher affinity for a particular polypeptide than the affinity for other polypeptide under the conditions generally employed by those of ordinary skill in the art when using the antibody. In the present invention, as “other polypeptide”, for example, normal human PCA-1 polypeptide and the like can be mentioned.

The antibody includes, but is not limited to, natural type antibodies such as polyclonal antibody, monoclonal antibody (mAb) and the like, chimera antibody, humanized antibody and single strand antibody produced by genetically modification technique, human antibody produced using human antibody producing transgenic animal and the like, antibody fragment prepared from Fab expression library, and binding fragments thereof. Preferably, the antibody is a polyclonal antibody, a monoclonal antibody or a binding fragment thereof.

The binding fragment means a partial region of the aforementioned antibody, which is specifically exemplified by F(ab′)₂, Fab′, Fab, Fv (variable fragment of antibody), sFv, dsFv (disulphide stabilized Fv), dAb (single domain antibody) and the like (Exp. Opin. Ther. Patents, Vol. 6, No. 5, p. 441-456, 1996).

The class of the antibody is not particularly limited, and also encompasses an antibody having any isotype such as IgG, IgM, IgA, IgD or IgE and the like. Preferably, it is IgG or IgM and, in consideration of easy purification and the like, IgG is more preferable.

The antibody of the present invention can be obtained by administering the polypeptide and the like to a mammal (preferably mammal other than human) such as mouse, rabbit and the like following a conventional protocol and the like.

A monoclonal antibody can be prepared according to the method of Köhler and Milstein et al. (Nature, Vol. 256, p. 495-497, 1975) or a method analogous thereto. In screening for a hybridoma producing an antibody that specifically reacts with the polypeptide of the present invention, a corresponding control peptide wherein a mutated site contained in the polypeptide of the present invention has been substituted for the amino acid of normal PCA-1 is used and a clone reactive with the polypeptide of the present invention but unreactive with the corresponding control peptide can be selected.

For preparation of the polyclonal antibody, the serum of an animal immunized with the-polypeptide of the present invention is preferably purified by a column conjugated with the polypeptide of the present invention and the like.. More preferably, a fraction capable of reacting with normal human PCA-1 is removed using a column conjugated with the above-mentioned corresponding control peptide, thereby to enhance the specificity;

The antibody of the present invention is preferably isolated or purified by saturated ammonium sulfate, eugloblin precipitation, caproic acid method, caprylic acid method, ion exchange chromatography (DEAE, DE52 etc.), or affinity column chromatography using anti-immunoglobulin column, protein A column, column where immunogen is crosslinked and the like.

Since the antibody of the present invention immunospecifically recognizes a mutated human PCA-1 polypeptide, for example, it is possible to analyze whether or not a PCA-1 gene derived from a subject has a mutation by Western blotting, immunohistological stain, ELISA method, RIA method, surface plasmonresonance method, protein chip and the like.

The method for determining a subject having a risk of having developed or developing prostate cancer of the present invention also encompasses a method using the above-mentioned antibody.

Moreover, the present invention relates to a kit for determining a subject having a risk of having developed or developing prostate cancer, which comprises a reagent for PCA-1 gene mutation analysis.

The kit further preferably comprises a written matter stating that the reagent for PCA-1 gene mutation analysis can or should be used for determining a subject having a risk of having developed or developing prostate cancer.

The reagent for PCA-1 gene mutation analysis may be any of the reagents used for the method of the present invention. For example, an oligonucleotide primer set used for amplification of PCA-1 gene, an isolated or purified polynucleotide already clarified to contain a normal PCA-1 gene, an isolated or purified polynucleotide already clarified to contain a mutated PCA-1 gene, an antibody immunospecifically bound to a mutated human PCA-1 polypeptide and the like can be mentioned.

The present invention is explained in more detail in the following by referring to Examples shown below, which are not to be construed as limitative.

### EXAMPLE 1

The postoperative specimens from four prostate cancer patients who had never undergone a chemical therapy or hormone therapy were histopathologically divided into cancerous part and peripheral non-cancerous part to obtain tissue samples. In addition, human prostate cancer cell lines (PC-3, DU145, LN-CaP) were also used.

The obtained tissues and cells were frozen and subjected to total RNA extraction. Frozen samples were homogenized in a TRIzol reagent (manufactured by Life Technologies, Inc., Rockville, Md., USA), and total RNA was isolated according to the protocol provided by the manufacturer. Potentially contaminating DNA was removed by a treatment with RNase-free DNaseI (manufactured by Wako Pure Chemical Industries, Ltd.). Reverse transcription reaction was carried out using total RNA (5 μg), oligo dT primer and AMV reverse transcriptase (manufactured by Life Technologies, Inc.). PCA-1 gene was amplified by PCR using a part of the obtained cDNA. The composition of the reaction mixture used for PCR was the above-mentioned cDNA (20 ng), KOD dash (manufactured by TOYOBO), DNTP (0.2 M each), 10× KOD dash buffer (2.5 μl), primer 1 (SEQ ID; No 4: 0.2 μM) and primer 2 (SEQ ID; No 5: 0.2 μM) in 25 μl PCR buffer. The reaction was carried out using a DNA Thermal Cycler (DNA Thermal Cycler 460, manufactured by PerkinElmer Inc.) under the conditions of 35 cycles of 95° C. 30 sec, 60° C. 30 sec and 72° C. 1 min 30 sec, and finally 72° C. 10 min. The reaction mixture was applied to agarose gel electrophoresis, and an amplification product was separated. A band containing the amplification product was isolated and the amplification product was purified by Concert (manufactured by Life Technologies, Inc.). The amplification product was subcloned to pT7-vector (manufactured by Novagen), and the base sequence was determined by a PRISM 310 DNA sequencer (manufactured by PerkinElmer).

The determined base sequence was compared with the base sequence shown in SEQ ID; No 1, and the amino acid sequence redicted from the determined base sequence and the sequence shown in SEQ ID; No 2 were compared, based on which the presence/absence of mutation was analyzed. The results are shown in Table 1.
L(SEQ ID; No 4): CTGAAAGCTCGGAGCAGAAGC (corresponding 373rd-393rd residues in SEQ ID; No 1)

2(SEQ ID; No 5): GGTCTACTGTGGGAACAG (corresponding to 42nd-1459th residues in SEQ ID; No 1)

TABLE 1

| Sample | Prostate | | Amino acid | |
|---|---|---|---|---|
| No. | cancer | Mutation sites | substitution | Kind of mutation |
| 1 | + | G426T, | Arg7Leu, | Mismatch mutation |
| | | C429T, | Ala8Val | Silent mutation |
| | | G1132T | | |
| 2 | + | del (777-865), | — | Flame shift |
| | | G1132T | | mutation due to |
| | | | | deletion |
| | | | | Silent mutation |
| 3 | + | G494A, G623A, | Ala30Thr, | Mismatch mutation |
| | | G815A, T836C, | Asp73Asn, | Silent mutation |
| | | A1189T, | Ser144Pro, | |
| | | G1132T | Gly137Arg, | |
| | | | Lys261Asn | |
| 4 | + | A1187T, | Lys261* | Nonsense mutation |
| | | T568C, G1132T | | Silent mutation |
| 5 | + | C528T, C1090G | Thr41Ile, | Mismatch mutation |
| | | | Asp228Glu | |
| 6 | + | C1090G | Asp228Glu | Mismatch mutation |
| 7 | + | C1090G, | Asp228Glu, | Mismatch mutation |
| | | A1105T | Glu233Asp | |

In Table 1, “Ala30Thr” means “mutation of the 30th to threonine”, “Lys261*n means “mutation of the 261st to a stop codon”, “T568C” means “mutation of the 568th thymine to cytosine” and “del(777-865)” means “deletion of the 777th-865th residues”.

In addition, sample Nos. 1-4 show cancer parts of prostate cancer patients, sample No. 5 shows LN-CaP cells, sample No. 6 shows PC-3 cells and sample No. 7 shows DU145 cells.

As is clear from Table 1, mutation of PCA-1 gene was observed in all prostate cancer samples (prostate cancer patients: 4/4, prostate cancer cell lines: 3/3). While the mutation sites and kinds thereof varied, mutation of G1132T was observed in all prostate cancer patients (4/4), and mutation of C1090G was observed in all prostate cancer lines (3/3), which accompanied amino acid substitution of Asp228Glu. Mutation of the 261st lysine was observed at comparatively high frequency (2/7). Most samples showed plural mutations (prostate cancer patients: 4/4, prostate cancer cell lines: 2/3).

Moreover, mutation causing amino acid mutation of the dealkylation enzyme active site of PCA-1 was observed at extremely high frequency (prostate cancer patients: 3/4, prostate cancer cell lines: 3/3). This suggests the relationship between abnormality in the dealkylation enzyme activity of PCA-1 due to the mutation of PCA-1 gene and the onset, progress etc. of prostate cancer.

In contrast, the above-mentioned mutations in the PCA-1 gene were not observed in non-cancer parts (3 persons/3 persons).

### EXAMPLE 2

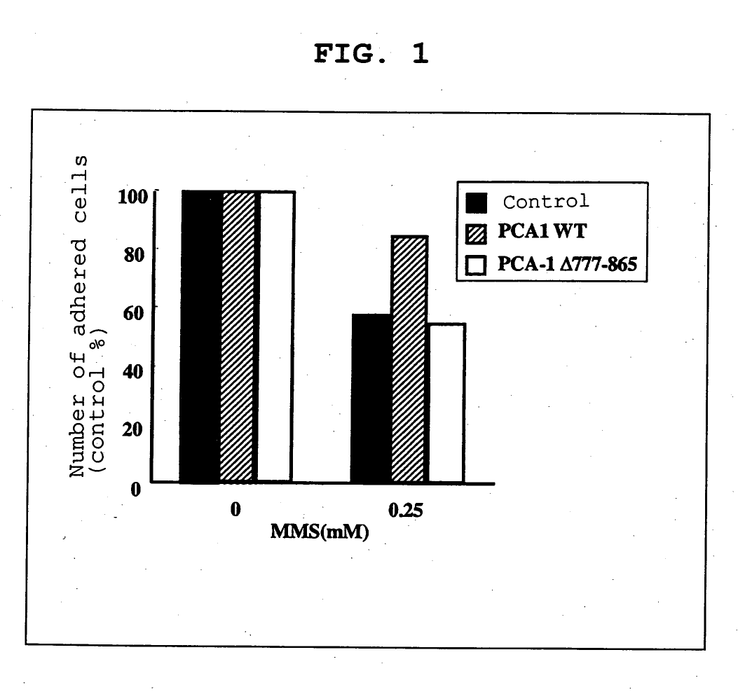

Normal PCA-1 (PCA-1WT) gene, and the PCA-1 deletion mutant (PCA-1A777-865), wherein the 777th-865th bases had been deleted, were incorporated into a mammal expression vector (PEGFP). These expression vectors (5 μg each) were transfected into COS-7 cells (1×10⁶) by the DEAE-Dextran method. In a control group, an empty expression vector was transfected into the cells. At 48 hr of transfection, 0.25 mM methyl methanesulfonate (MMS) was added to induce alkylation damage of the nucleic acid, and at 24 hr thereafter, the number of adhered cells was measured. The results are shown in FIG. 1.

As is clear from FIG. 1, the death of about 40% of the cells due to the alkylation damage of nucleic acid caused by 0.25 mM MMS was observed in the control group and the PCA-1 ₆₆ 777-865 transfection group. In contrast, the MMS-induced alkylation damage was remarkably reduced in the PCA-1WT transfection group. From the results, it was shown that normal PCA-1 expressed in vivo dealkylation activity to alkylated DNA but PCA-1_{Δ}777-865 lacked the activity. In addition, it was shown that mutation in the dealkylation enzyme active site of PCA-1 could cause abnormality in the dealkylation activity.

### INDUSTRIAL APPLICABILITY

According to the determination method of prostate cancer of the present invention, a subject having a risk of having developed or developing prostate cancer can be determined conveniently and highly sensitively. Therefore, the method is effective for the diagnosis of prostate cancer, progress monitoring, prognostic prediction, diagnosis before the onset, carrier diagnosis and the like.

This application is based on a patent application No. 2004-47036 filed in Japan, the contents of which are incorporated in full herein by this reference.

### Sequence Listing Free Text

SEQ ID; No 4: oligonucleotide designed to act as a PCR primer for detecting PCA-1 gene

SEQ ID; No 5: oligonucleotide designed to act as a PCR primer for detecting PCA-1 gene

1. A method for determining a subject having a risk of having developed or developing prostate cancer, which comprises the following steps:
a) a step of analyzing the presence/absence or level of mutation in the PCA-1 gene derived from the subject; and
b) a step of evaluating the presence/absence or level of the subject's risk of having developed or developing prostate cancer, based on the presence/absence or level of mutation in the PCA-1 gene.

2. The method of claim 1, wherein the mutation is selected from the group consisting of a missense mutation, a nonsense mutation, a silent mutation and a frame shift mutation due to deletion.

3. The method of claim 2, wherein the missense mutation causes an amino acid mutation selected from the group consisting of mutation of the 7th arginine to leucine, mutation of the 8th alanine to valine, mutation of the 30th alanine to threonine, mutation of the 41st threonine to isoleucine, mutation of the 73rd aspartic acid to asparagine, mutation of the 137th glycine to arginine, mutation of the 144th serine to proline, mutation of the 228th aspartic acid to glutamic acid, mutation of the 233rd glutamic acid to aspartic acid, and mutation of the 261st lysine to asparagines, in PCA-1 polypeptide.

4. The method of claim 3, wherein the missense mutation causes an amino acid mutation selected from the group consisting of mutation of the 228th aspartic acid to glutamic acid, mutation f the 233rd glutamic acid to aspartic acid, and mutation of he 261st lysine to asparagines, in PCA-1 polypeptide.

5. The method of claim 4, wherein the missense mutation causes a mutation of the 228th aspartic acid to glutamic acid in PCA-1 polypeptide.

6. The method of claim 3, wherein the missense mutation is selected from the group consisting of mutation of the 426th guanine to thymine, mutation of the 429th cytosine to thymine, mutation of the 494th guanine to adenine, mutation of the 528th cytosine to thymine, mutation of the 623rd guanine to adenine, mutation of the 815th guanine to adenine, mutation of the 836th thymine to cytosine, mutation of the 1090th cytosine to guanine, mutation of the 1105th adenine to thymine, and mutation of the 1189th adenine to thymine, in PCA-1 gene.

7. The method of claim 6, wherein the missense mutation is selected from the group consisting of mutation of the 1090th cytosine to guanine, mutation of the 1105th adenine to thymine, and mutation of the 1189th adenine to thymine, in PCA,1 gene.

8. The method of claim. 7, wherein the missense mutation is mutation of the 1090th cytosine to guanine in PCA-1 gene.

9. The method of claim 2, wherein the nonsense mutation causes mutation of the 261st lysine to a stop codon in PCA-1 polypeptide.

10. The method of claim 9, wherein the nonsense mutation is mutation of the 1187th adenine to thymine in PCA-1 gene.

11. The method of claim 2, wherein the silent mutation is selected from the group consisting of mutation of the 568th thymine to cytosine and mutation of the 1132nd guanine to thymine, in PCA-1 gene.

12. The method of claim 11, wherein the silent mutation is mutation of the 1132nd guanine to thymine in PCA-1 gene.

13. The method of claim 2, wherein the deletion is a deletion of the residue from the 777th adenine to the 865th cytosine in PCA-1 gene.

14. The method of claim 1, which comprises the following steps:
a) a step of isolating or purifying a polynucleotide from the subject;
b) a step of amplifying a PCA-1 gene from the polynucleotide;
c) a step of determining the base sequence of the amplified PCA-1 gene;
d) a step of analyzing the presence/absence or level of mutation in the PCA-1 gene derived from the subject; ‘e) a step of evaluating the presence/absence or level of the subject's risk of having developed or developing prostate cancer, based on the presence/absence or level of mutation in the PCA-1 gene.

15. A polynucleotide comprising a base sequence having not less than one substitution or deletion selected from the group consisting of substitution to the 426th guanine to thymine, substitution of the 429th cytosine by thymine, substitution of the 494th guanine by adenine, substitution of the 528th cytosine by thymine, substitution of the 623rd guanine by adenine, substitution of the 815th guanine by adenine, substitution of the 836th thymine by cytosine, substitution of the 1090th cytosine by guanine, substitution of the 1105th adenine by thymine, substitution of the 1189th adenine by thymine, substitution of the 1187th adenine by thymine, substitution of the 568th thymine by cytosine, substitution of the 1132nd guanine by thymine, and deletion of a sequence from the 777th adenine to the 865th cytosine, as compared to SEQ ID; No 1.

16. A polypeptide comprising an amino acid sequence encoded by a base sequence having not less than one substitution or deletion selected from the group consisting of substitution to the 426th guanine to thymine, substitution of the 429th cytosine by thymine, substitution of the 494th guanine by adenine, substitution of the 528th cytosine by thymine, substitution of the 623rd guanine by adenine, substitution of the 815th guanine by adenine, substitution of the 836th thymine by cytosine, substitution of the 1090th cytosine by guanine, substitution of the 1105th adenine by thymine, substitution of the 1189th adenine by thymine, substitution of the 1187th adenine by thymine, and deletion of a sequence from the 777th adenine to the 865th cytosine, as compared to SEQ ID; No 1.

17. An antibody immunospecifically recognizing the polypeptide of claim 16.

18. A kit for determining a subject having a risk of having developed or developing prostate cancer, which comprises a reagent for PCA-1 gene mutation analysis.

19. The kit of claim. 18, further comprising a written matter associated therewith, the written matter stating that the reagent for PCA-1 gene mutation analysis can or should be used for determining a subject having a risk of having developed or developing prostate cancer.


SEQUENCE LISTING (5 sequences)

SEQ ID NO: 1 - DNA, 1520 bp, Homo sapiens
  CDS (407)..(1267)
aggtcacagactgcggagtgggtcaggggctgcgagggctgccccaagtcctaccgggtttgcacgggcgcgcccggctccgcccgcaagtgcgccttcctgacttactgctgggtgcgcggggctgggggtgcgagtaccacccctgaagtctcttcctgggcgacctccggggcctcattctaggcctccttaaagagaaggatctaaattaggaaaaggaagtgcccttatccacgaccaagctcttccacctgcggagctcgcttagtctgcacctcaaccgtgcggaaagtgactgccctgtttactgaggaaaaactggggctcagaaagataccatggagtagtttgaaacaggaacaaaatcttctgaaagctcggagcagaagcctttttggtcaacatggaggaaaaaagacggcgagcccgagttcagggagcctgggctgcccctgttaaaagccaggccattgctcagccagctaccactgctaagagccatctccaccagaagcctggccagacctggaagaacaaagagcatcatctctctgacagagagtttgtgttcaaagaacctcagcaggtagtacgtagagctcctgagccacgagtgattgacagagagggtgtgtatgaaatcagcctgtcacccacaggtgtatctagggtctgtttgtatcctggctttgttgacgtgaaagaagctgactggatattggaacagctttgtcaagatgttccctggaaacagaggaccggcatcagagaggatataacttatcagcaaccaagacttacagcatggtatggagaacttccttacacttattcaagaatcactatggaaccaaatcctcactggcaccctgtgctgcgcacactaaagaaccgcattgaagagaacactggccacaccttcaactccttactctgcaatctttatcgcaatgagaaggacagcgtggactggcacagtgatgatgaaccctcactagggaggtgccccattattgcttcactaagttttggtgccacacgcacatttgagatgagaaagaagccaccaccagaagagaatggagactacacatatgtggaaagagtgaagatacccttggatcatgggaccttgttaatcatggaaggagcgacacaagctgactggcagcatcgagtgcccaaagaataccactctagagaaccgagagtgaacctgacctttcggacagtctatccagaccctcgaggggcaccctggtgacgtcagagctttgagagagaagcttcactgaaacggagcaaaccttccactgagaagccacttcaagaggctggtgctgctagatctcatgatgtggctgttgggaagatggtggggtttgtttgccagcttggagtcctattaaatgaaagccagcaactcatgttggtaataggtctactgtgggaacagttatccctaaccacagctcaaaatcgctatcatctttaggcaaattaaaatctatgtggca

SEQ ID NO: 2 - protein, 286 aa, Homo sapiens
MEEKRRRARVQGAWAAPVKSQAIAQPATTAKSHLHQKPGQTWKNKEHHLSDREFVFKEPQQVVRRAPEPRVIDREGVYEISLSPTGVSRVCLYPGFVDVKEADWILEQLCQDVPWKQRTGIREDITYQQPRLTAWYGELPYTYSRITMEPNPHWHPVLRTLKNRIEENTGHTFNSLLCNLYRNEKDSVDWHSDDEPSLGRCPIIASLSFGATRTFEMRKKPPPEENGDYTYVERVKIPLDHGTLLIMEGATQADWQHRVPKEYHSREPRVNLTFRTVYPDPRGAPW

SEQ ID NO: 3 - DNA, 861 bp, Homo sapiens
atggaggaaaaaagacggcgagcccgagttcagggagcctgggctgcccctgttaaaagccaggccattgctcagccagctaccactgctaagagccatctccaccagaagcctggccagacctggaagaacaaagagcatcatctctctgacagagagtttgtgttcaaagaacctcagcaggtagtacgtagagctcctgagccacgagtgattgacagagagggtgtgtatgaaatcagcctgtcacccacaggtgtatctagggtctgtttgtatcctggctttgttgacgtgaaagaagctgactggatattggaacagctttgtcaagatgttccctggaaacagaggaccggcatcagagaggatataacttatcagcaaccaagacttacagcatggtatggagaacttccttacacttattcaagaatcactatggaaccaaatcctcactggcaccctgtgctgcgcacactaaagaaccgcattgaagagaacactggccacaccttcaactccttactctgcaatctttatcgcaatgagaaggacagcgtggactggcacagtgatgatgaaccctcactagggaggtgccccattattgcttcactaagttttggtgccacacgcacatttgagatgagaaagaagccaccaccagaagagaatggagactacacatatgtggaaagagtgaagatacccttggatcatgggaccttgttaatcatggaaggagcgacacaagctgactggcagcatcgagtgcccaaagaataccactctagagaaccgagagtgaacctgacctttcggacagtctatccagaccctcgaggggcaccctggtga

SEQ ID NO: 4 - DNA, 21 bp, Artificial
  Oligonucleotide designed to act as PCR primer for detection of PCA-1 gene
ctgaaagctcggagcagaagc

SEQ ID NO: 5 - DNA, 18 bp, Artificial
  Oligonucleotide designed to act as PCR primer for detection of PCA-1 gene
ggtctactgtgggaacag